(19) United States
(12) Patent Application Publication
(10) Pub. No.: US 2025/0020625 A1
(43) Pub. Date: Jan. 16, 2025

(54) RAPID LOAD TEST METHOD FOR PILE BY USE OF SEGMENTAL UNLOADING POINT CONNECTION METHOD

(71) Applicant: JIBANSHIKENJO CO., LTD., Tokyo (JP)

(72) Inventors: Isaku YAMAMOTO, Tokyo (JP); Shuichi KAMEI, Tokyo (JP); Tatsunori MATSUMOTO, Tokyo (JP); Go KOBAYASHI, Tokyo (JP); Shihchun LIN, Tokyo (JP)

(21) Appl. No.: 18/549,417

(22) PCT Filed: Dec. 6, 2022
(86) PCT No.: PCT/JP2022/044886

§ 371 Date: Sep. 7, 2023

(51) Int. Cl. G01N 33/24; G01L 5/16; G01N 3/303

(52) U.S. Cl. CPC G01N 33/24; G01L 5/16; G01N 3/303

(57) ABSTRACT

Disclosed is a rapid load test method for a pile by use of a method resulting from extending a well-known SULP method, wherein a rapid load test is implemented on a pile head more than once in the manner of changing a drop height h of a weight, with the pile instrumented with strain gages and accelerometers at more than one depth level, followed by the processing of obtaining a rapid load F_{rapid}-pile displacement w relation for each pile segment for each time of the rapid load test using measurement data measured by the strain gages and the accelerometers, of obtaining a soil resistance Rₛₒᵢₗ-pile displacement w relation on each pile segment for each time of the rapid load test, of calculating an unloading point that can be a point of maximum displacement of the pile, together with an unloading point load R_{ULP} at that moment, and of obtaining a static soil resistance R_{w}-pile displacement w relation on each pile segment by use of an Unloading Point Connection method using the calculated unloading point and unloading point load R_{ULP} for each time of the rapid load test. Accordingly, an analysis on a load-displacement relation is performed by use of a load transfer method with respect to the whole pile, using a nonlinear model of the thus obtained soil resistance on each pile segment.

### TECHNICAL FIELD

This invention relates to a rapid load test method for a pile, in which a weight is dropped onto a pile head to determine a static load-displacement relation of the pile from a relation between a load caused by drop of the weight and a displacement of the pile head, more specifically, to a rapid load test method for a pile, with which a more highly precise load-displacement relation can be obtained using a newly developed analysis method, in relation to an analysis method for determining a load-displacement relation of the pile on condition that a rapid load test is implemented on a pile head more than once in the manner of changing a weight drop height h, with the pile instrumented with strain gages and accelerometers at more than one depth level.

### BACKGROUND ARTS

As some patent documents on a rapid load test method for a pile/an analysis method in the prior arts, the following patent documents 1 to 3 can be given, for instance.

In relation to a rapid load test method for determining pile settlement stiffness and pile bearing capacity by dropping a weight onto a pile head to apply striking force to the pile head, the patent document 1 below discloses a technique that makes it possible to obtain a highly precious test result with a smaller load in such a way that a cushion material made of a material with specific gravity in the range of not less than 0.35 to not more than 0.5 is interposed between the weight and the pile head.

As one rapid load test equipment for a pile for obtaining highly reliable data simply in a short period of time, as data necessary for estimation of bearing capacity of the pile driven into the ground, the patent document 2 below discloses the rapid load test equipment for a pile, in which a cushion material formed by laminating two or more metal plates and a polymer material sandwiched between these metal plates is interposed between a pile head and a weight for applying a blow to the pile head.

The patent document 3 below discloses the rapid load test equipment for a pile for obtaining highly reliable data simply in a short period of time in such a way that a weight dropped onto a pile head and subsequently bounding therefrom is made to quickly catch so as to discontinue rebounding of the weight, in cases where a cushion material is interposed between the pile head and the weight for applying a blow to the pile head.

In Japan, as to the rapid load test method for the pile as described the above, it has been 20 years since rapid load testing on piles was incorporated, as one dynamic load test method, in the load testing standards (JGS1815-2002) of Japanese Geotechnical Society. Up to 2002 when the rapid load testing on the piles was incorporated in the load testing standards, an inertial force method using a reaction body had been dominantly adopted for the force application equipment for rapid load tests, whereas as of this moment with advanced improvements on the force application equipment, most of rapid load tests in Japan are being implemented with a weight drop method using a soft cushion.

Accordingly, in regards to the number of times of loading to be performed, there has been a tendency to adopt loading that is performed more than once in the manner of heightening a hammer drop height in steps, in place of a loading method in which loading of a design maximum load is performed once. For that reason, it has been a mainstream for an analysis method to adopt an Unloading Point Connection (ULPC) method that is capable of obtaining a static load-displacement relation only by connecting unloading points without any need to determine a damping constant C required for an Unloading Point (ULP) method, in place of the Unloading Point (ULP) method.

As a result of the spread of the Unloading Point Connection (ULPC) method as one analysis method for the rapid load tests in this way, there have been some cases where the Unloading Point Connection method becomes an issue in terms of an analysis with a single-mass system model in which the whole pile in a length direction is assumed to be a rigid body. Therefore, several different analysis methods such as a method using a mean of accelerations measured by more than one accelerometer instrumented on a pile body and a method in which an acceleration level is reduced to that corresponding to the magnitude of an acceleration at a pile tip, for instance, have been proposed for use as a basis for estimation on inertial forces when being judged that no behavior is observed in a single-mass system.

Meanwhile, non-patent document 1 below proposes a SULP method as one analysis method that takes into account the estimation on inertial forces. FIG. 1 is a conceptual view of the SULP method.

The SULP method is a method that is applicable to an analysis in cases where the pile is instrumented with strain gages at more than one depth level. The SULP method is supposed to set pile segments at depth levels corresponding to positions where the pile is instrumented with the strain gages, and an Unloading Point method is applied to obtain a rapid load and a displacement for every pile segment. After that, segmental static resistances obtained from the analysis results are summed up.

### PRIOR ART DOCUMENTS

### Patent Documents

Patent document 1: Japanese Unexamined Patent Application Publication No. 2002-303570
Patent document 2: Japanese Unexamined Patent Application Publication No. 2005-068802
Patent document 3: Japanese Patent Publication No. 6613430

### Non-Patent Document

Non-patent document 1: Gray Mullins et al. (2002): Advancements in Statnamic data regression techniques, International Deep Foundations Congress, Orlando, Florida, USA, pp. 1 to 16.

### SUMMARY OF THE INVENTION

### Problems to be Solved by the Invention

With the SULP method as described the above, a way of partitioning the pile into segments can reduce a length of the pile to be analyzed, even when differences in the magnitude of acceleration, velocity and displacement and/or time differences arise over the whole pile. Therefore, this SULP method provides the advantages of being made more approximate to a condition of assuming the pile to be the rigid body, which is one analysis condition of the Unloading Point method, and of being capable of calculating both segmental pile inertial forces and soil resistances that are close to the actual phenomenon.

However, because of the resistances obtained at different times, namely, by different segmental pile displacements, a way of simply summing up the segmental static resistances to calculate a pile head load may be attributed to overestimation of the bearing capacity.

Besides, for the SULP method, the segmental pile displacements are measured using the strain gages, and the accelerations are determined by means of second-order differentiation thereof, with the result that estimation on the inertial forces may be affected by errors due to the differentiation.

The present invention proposes a Segmental Unloading Point Connection method (which will be referred to as “SULPC method”) that is an extended SULP method, as a newly developed analysis method to solve the above problems, with the object of providing a rapid load test method for a pile, with which it is possible to perform quantitative estimation on inertial forces, thus allowing a more highly precise load-displacement relation to be obtained, even when differences in the magnitude of acceleration and/or time differences arise over the whole pile.

### Means for Solving the Problems

In a rapid load test method for a pile, in which a weight is dropped onto a pile head to determine a load-displacement relation of the pile from both of a load caused by drop of the weight and a displacement of the pile head, a rapid load test method for a pile by use of a Segmental Unloading Point Connection method of the present invention is characterized in that a load-displacement relation of the pile is determined in accordance with the following steps on condition that a rapid load test is implemented on the pile head more than once in the manner of changing a drop height h of the weight, with the pile instrumented with strain gages and accelerometers at more than one depth level.

Step of, in the rapid load test implemented more than once, obtaining a rapid load F_{rapid}-pile displacement w relation on each pile segment, for each time of the rapid load test, using measurement data measured by the strain gages and accelerometers at more than one depth level of the pile,

(2) Step of, for each time of the rapid load test, obtaining a soil resistance Rₛₒᵢₗ-pile displacement w relation on each pile segment, followed by the processing of calculating an unloading point that can be a point of maximum displacement of the pile, together with an unloading point load R_{ULP} at that moment,

(3) Step of obtaining a static soil resistance R_{w}-pile displacement w relation on each pile segment by use of an Unloading Point Connection method, using the calculated unloading point and unloading point load R_{ULP} for each time of the rapid load test in the above Step (2), followed by the processing of performing modeling of the obtained static soil resistance-pile displacement relation into a nonlinear model, and

(4) Step of performing an analysis on a load-displacement relation by use of a load transfer method with respect to the whole pile, using the obtained nonlinear model of soil resistance on each pile segment in the above Step (3).

As previously described, the analysis method (the SULPC method) in the present invention is the extended SULP method, and therefore can be applied to the analysis in the cases where the pile body is instrumented with the strain gages and the accelerometers at more than one depth level.

As shown in the conceptual view of FIG. 2 and also by the following equation (1), the pile is partitioned into segments at positions where the pile is instrumented with the strain gages and the accelerometers, so that the analysis shall be performed for every pile segment.

$$\begin{matrix}Equation 1 & \\ {R}_{soili} ( t ) = {R}_{wi} ( t ) + {R}_{vi} ( t ) = {F}_{rapidi} ( t ) - {m}_{i} \cdot {α}_{i} ( t ) = {F}_{i - 1} ( t ) - {F}_{i} ( t ) - {m}_{i} \cdot {α}_{i} ( t ) & ( 1 )\end{matrix}$$

In FIG. 2 and the equation (1), n is number of pile segments, i is segment, Rₛₒᵢₗᵢ is soil resistance on i-segment, R_{wi} is static soil resistance component on i-segment, Rᵥᵢ is dynamic soil resistance component on i-segment, F_{rapidi} is rapid load on i-segment, Fᵢ is rapid load in i-segmental cross-section, mᵢ is mass of i-segmental pile body, and αᵢ is acceleration of i-segmental pile body.

The analysis shall be performed by the procedure of firstly calculating the unloading points from both the soil resistance on each pile segment and the segmental pile displacement measured by the accelerometer, followed by the processing of a determining static load-displacement relation and/or axial force distributions by use of the load transfer method in such a way as to use the calculated unloading points to calculate the static soil resistance-displacement relation on each pile segment, likewise a well-known Unloading Point Connection (ULPC) method. A processing flow of the analysis is shown in FIG. 3, and images of the analysis results are shown in FIGS. 4 to 6. The analysis method (SULPC method) in the present invention has the following features.

By contrast to the SULP method in which the static soil resistance-displacement relation is determined from the result of a single blow by use of the Unloading Point method, the analysis method (SULPC method) in the present invention is supposed to determine the load-displacement relation on each pile segment by connecting a plurality of unloading points obtained from the test results, therefore allowing initial settlement stiffness and/or a load-displacement relation up to that for a maximum resistance to be obtained without any need to determine a damping constant C.

The analysis method (SULPC method) in the present invention is premised on the pile body instrumented with more than one accelerometer. Therefore, considering a concept that the pile shall be assumed to be the rigid body, which is one concept that the SULP method features, it is possible for the analysis method (SULPC method) to obtain both the pile inertial force and the load-displacement relation on each pile segment with great precision.

The analysis method (SULPC method) in the present invention performs the processing of modeling the static soil resistance-displacement relation on each pile segment into a nonlinear spring model, therefore allowing a pile head load-pile head displacement relation and/or axial force distributions to be obtained by use of the load transfer method. By so doing, the static resistance corresponding to the pile body displacement caused at each depth level can be obtained, resulting in no need to simply sum up the segmental static resistances, unlike the SULP method.

In this way, for the analysis method (SULPC method) in the present invention, it is possible to perform the quantitative estimation on the inertial forces, thus allowing the pile head load-pile head displacement relation and/or the axial force distributions to be obtained with great precision, even when the differences in the magnitude of acceleration and/or the time differences arise over the whole pile.

The analysis method (SULPC method) in the present invention is supposed to calculate the unloading points for every segmented pile length as shown in FIG. 2. Accordingly, provided that a relative loading duration Tᵣ meets Tᵣ≥5 in the pile segment, this analysis method (SULPC method) can fall in the range in which the single-mass system model is covered, with the result that an effect of the wave phenomenon is made negligible. In other words, it is not necessary for the relative loading duration Tᵣ to meet Tᵣ≥5 over the whole length of the pile.

It is to be noted that the dynamic load test involves a rapid load test and an impulsive load test, and these two load tests are categorized according to whether or not waves generated in the pile body are made negligible. Categorizing in this manner is determined based on a length of the loading duration, and is thus defined in the frequency of round travels of a wave that is traveling through the inside of the pile body. The frequency of round travels of the wave through the inside of the pile during the loading duration can be the relative loading duration Tᵣ, wherein the relative loading duration is calculated based on a loading duration in which the duration of one round travel of the wave through the inside of the pile is assumed to be 1. The load test with a relative loading duration of less than 5 is categorized as the impulsive load test, the load test with a relative loading duration in the range of not less than 5 to not more than 500 is categorized as the rapid load test, and the load test with a relative loading duration of not less than 500 is categorized as the static load test.

The relative loading duration Tᵣ is expressed as Tᵣ=t_{L}/(2 L/c), wherein Tᵣ is relative loading duration, ti is loading duration, L is pile length, and c is longitudinal wave velocity.

### Effects of the Invention

By using the analysis method (SULPC method) proposed in the present invention, in relation to the analysis method for determining the load-displacement relation of the pile on condition that the rapid load test is implemented on the pile head more than once in the manner of changing the drop height h of the weight, with the pile instrumented with the strain gages and the accelerometers at more than one depth level, it is possible to perform the quantitative estimation on the inertial forces, thus allowing the more highly precise load-displacement relation to be obtained, even when the differences in the magnitude of acceleration and/or the time differences arise over the whole pile.

### BRIEF DESCRIPTION OF THE DRAWINGS

FIG. 1 is a conceptual view of a well-known SULP method.

FIG. 2 is a conceptual view of a newly developed analysis method (SULPC method) for use in the present invention.

FIG. 3 is a flowchart of the newly developed analysis method (SULPC method) for use in the present invention.

FIG. 4 is a graphical representation of a relation between a rapid load F_{rapid} on each pile segment and a segmental pile displacement w.

FIG. 5 is a graphical representation of a relation between soil resistance Rₛₒᵢₗ on each pile segment and a segmental pile displacement w, which is obtained from the result of a single blow.

FIG. 6 is a graphical representation of a relation between static soil resistance R_{w} on each pile segment and a segmental pile displacement w, which is obtained from the results of all the blows.

FIG. 7 shows a geological columnar section in the vicinity of a test pile, together with a state of penetration of the test pile.

FIG. 8 is a graphical representation of a loading cycle in an indentation test.

FIG. 9 is a graphical representation of a load-displacement relation obtained by use of the indentation test.

FIG. 10 is a graphical representation of time histories of rapid load, displacement, velocity and acceleration (h=3.0 m).

FIG. 11 is a graphical representation of a relation between soil resistance on each pile segment and a segmental pile displacement (h=3.0 m).

FIG. 12 is a graphical representation of a relation between static soil resistance on each pile segment and a segmental pile displacement w (h=3.0 m).

FIG. 13 is a graphical representation of a pile head load-pile head displacement relation.

FIG. 14 shows axial force distributions.

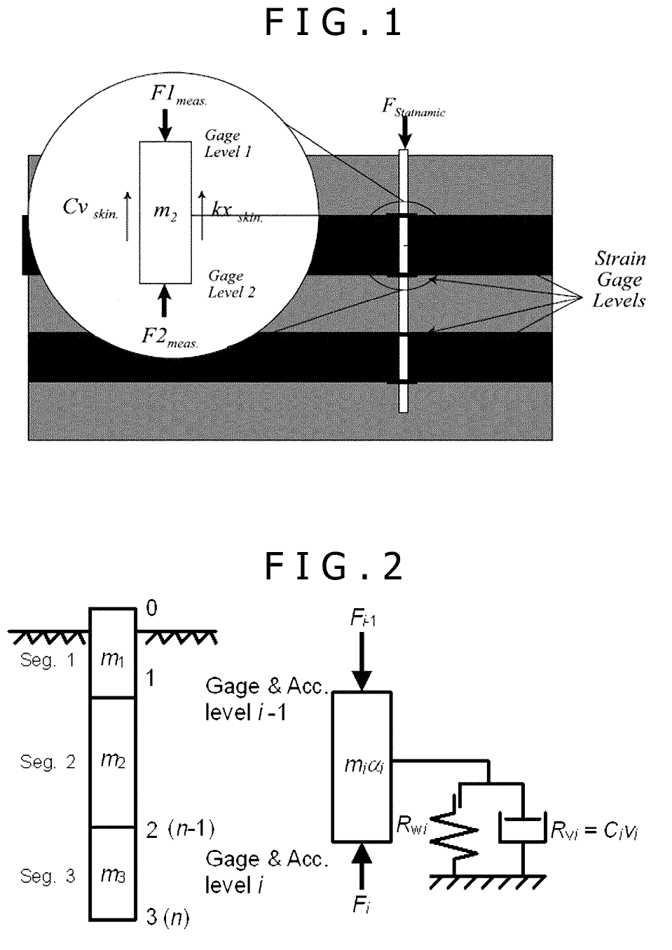

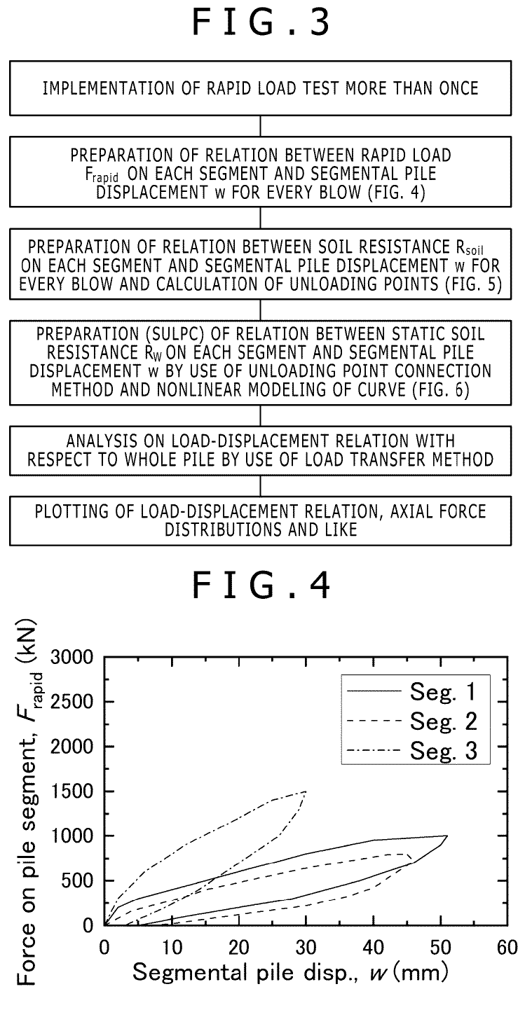

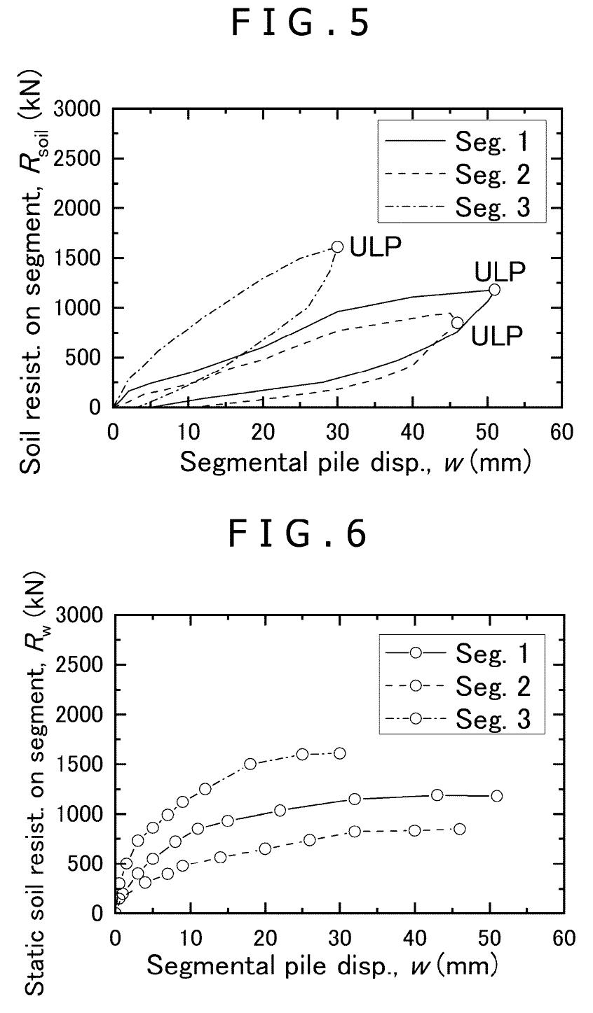

### MODE FOR EMBODYING THE INVENTION

Hereinafter will be described a test having been implemented for verification on the rationality of a rapid load test method according to the present invention. This test is to obtain the results of analysis on a load-displacement relation of a pile by use of an analysis method (SULPC method) in the present invention, with the object of weighing these analysis results against the indentation test results.

### [Test Pile Specification, Profiles of Soil Layers, and Load Testing Procedure]

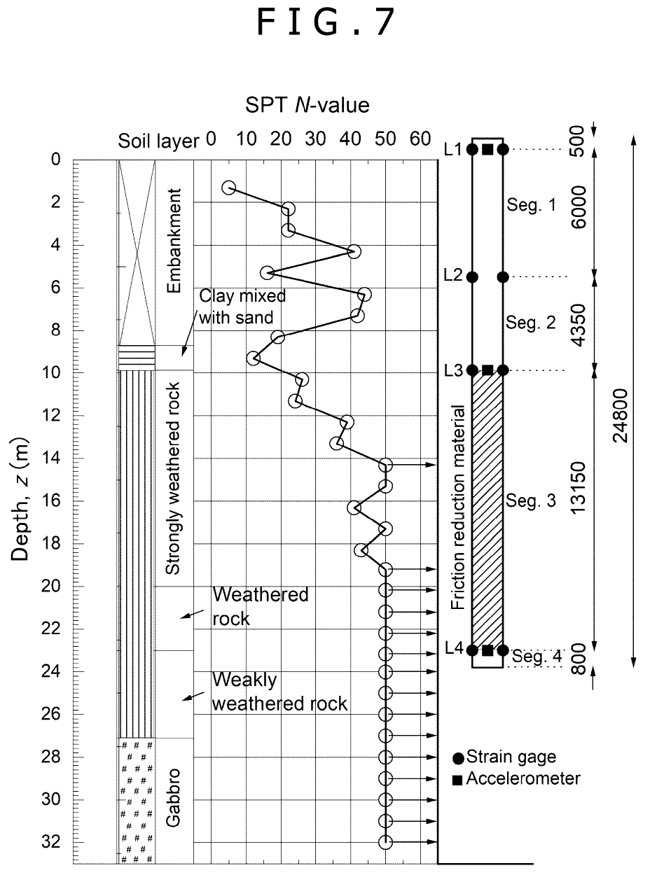

Table 1 below shows a test pile specification, and FIG. 7 shows a geological columnar section in the vicinity of a test pile, together with a penetration state of the test pile. A bearing stratum consists of weathered rock and strongly weathered rock, and N-values of the bearing stratum at a depth of not less than 20 m are 50 or more. Installation of the pile was performed by a Down-the-hole hammering construction method, in which case, a pile part ranging from the pile tip to 1D₀ segment was closed up by placement of foot protection concrete.

TABLE 1
Test pile specification

| Items | Value |
|---|---|
| Length, L (m) | 24.8 |
| Outer diameter, Dₒ (mm) | 800 |
| Inner diameter, Dᵢ (mm) | 772 |
| Wall thickness, t_{w} (mm) | 14 |
| Cross-sectional area, A (m²) | 0.0346 |
| Cross-sectional area, A (m²)* | 0.0376 |
| Young's modulus, E (kPa) | 2.00 × 10⁸ |
| Density, p (ton/m³) | 7.85 |
| Mass, m (ton) | 7.032 |
| Longitudinal wave velocity, c (m/s) | 5048 |

*Including a protection cover for strain gages

The pile was instrumented with both the strain gages and the accelerometers in the vicinity (L1) of the pile head. Meanwhile, for an underground part, the pile was instrumented with the strain gages at L2 to L4, and also with the accelerometers at L3 and L4. In each measurement point of the pile, two strain gages were placed in axially symmetrical positions. To allow a load on the pile head to be sufficiently transmitted to the pile tip, an outer pile surface at a pile segment between L3 and L4 in correspondence with weathered rock layer sections was coated with a friction reduction material, with the exception of a pile segment of 0.8 m from the pile tip.

As to the load tests for the pile, the indentation test was implemented after the lapse of a curing period of 29 days since the pile was installed, and the rapid load test followed after the lapse of 90 days since the indentation test was over.

### [Indentation Test Results]

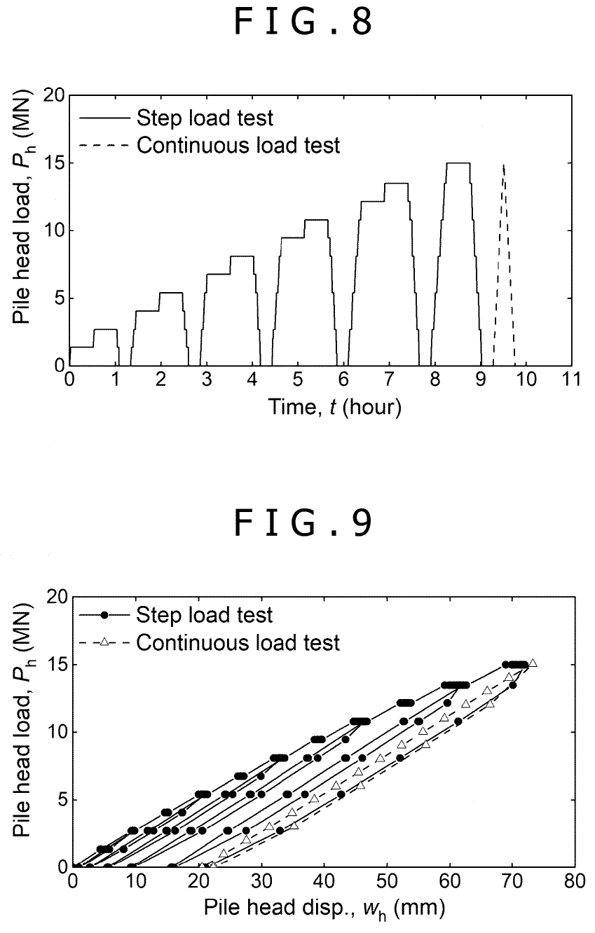

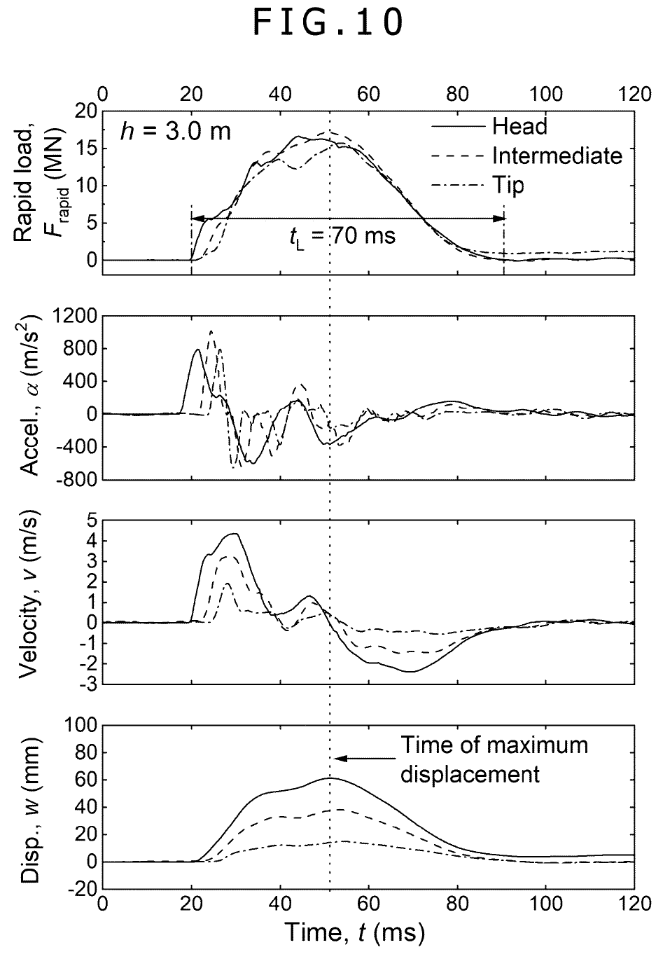

FIG. 8 shows a loading cycle in the indentation test. The indentation test was firstly implemented with a step load test method, followed by the indentation test with a continuous load test method. The maximum load for the continuous load test method was set to be equal to that for the step load test method. FIG. 9 shows a load-displacement relation obtained by use of the indentation test.

### [Rapid Load Test Results]

The rapid load test was implemented with a weight drop method using a soft cushion, together with a weight with a mass of 44 tons. This rapid load test was carried out seven times in total in the range of a hammer drop height h from h=0.25 to h=3.0 m.

FIG. 10 shows time histories of rapid load, displacement, velocity and acceleration when the maximum drop height h=3.0 m. It is obvious from FIG. 10 that the relative loading duration Tᵣ=t_{L}/(2 L/c) (where ti is loading duration) is 7.1 as much as the test criteria are satisfied. It is to be noted that the pile was instrumented with no accelerometer at L2, use was made of a weighted mean value obtained from measured acceleration data at L1 and L3 with distances from L1 and L3 to L2.

An analysis was performed by use of the analysis method (SULPC method) in the present invention in such a way as to partition the pile into segments (four segments) at L1 to L4 where the pile was instrumented with the strain gages, as shown in FIG. 7.

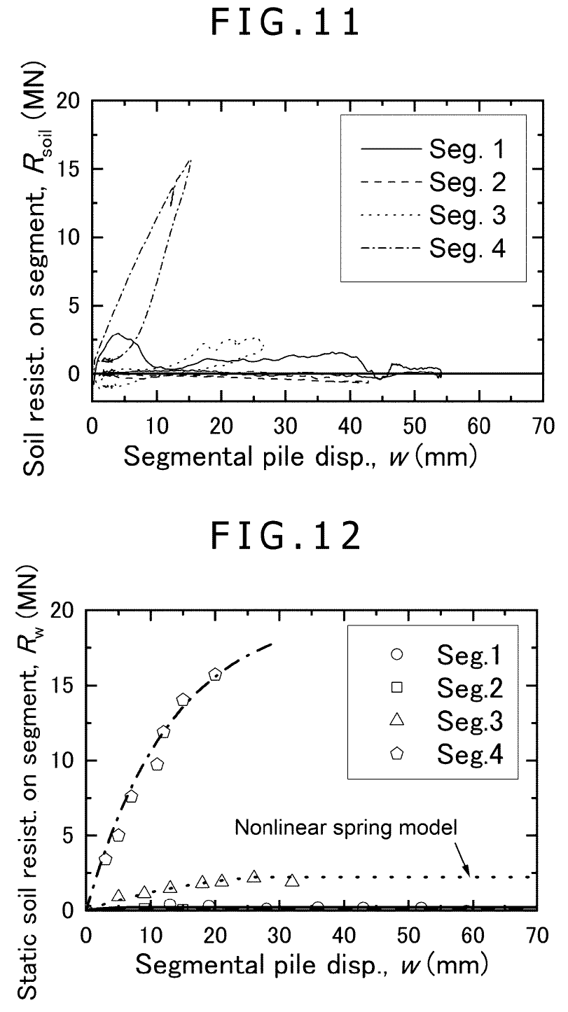

FIG. 11 shows a relation between soil resistance on each pile segment and a segmental pile displacement, which was obtained by use of the analysis method (SULCP method) in the present invention when the maximum drop height h=3.0 m. The unloading points were obtained from FIG. 11, followed by the processing of connecting the unloading points at respective drop heights by use of an Unloading Point Connection (ULPC) method to obtain a static soil resistance-pile displacement relation. The results thereof are shown in FIG. 12.

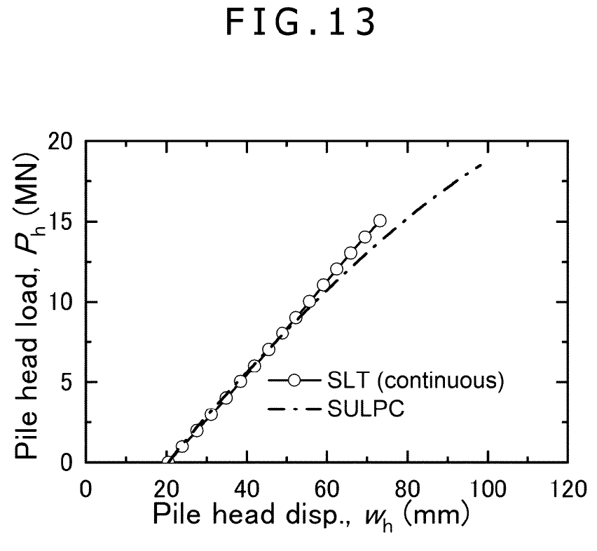

Thereafter, a pile head load-pile head displacement relation was obtained by use of a load transfer method in such a way as to perform modeling of the static soil resistance-pile displacement relation on each pile segment as shown in FIG. 12 into a nonlinear spring model. The results thereof are shown in FIG. 13. In FIG. 13, a load-displacement relation obtained by use of the continuous load test method is also shown together. A load-displacement curve resulting from the analysis by use of the analysis method (SULPC method) in the present invention exhibited the result being nearly in conformity with the indentation test result (the continuous load test method). In particular, it is found that the initial settlement stiffness was substantially in agreement with the continuous load test result.

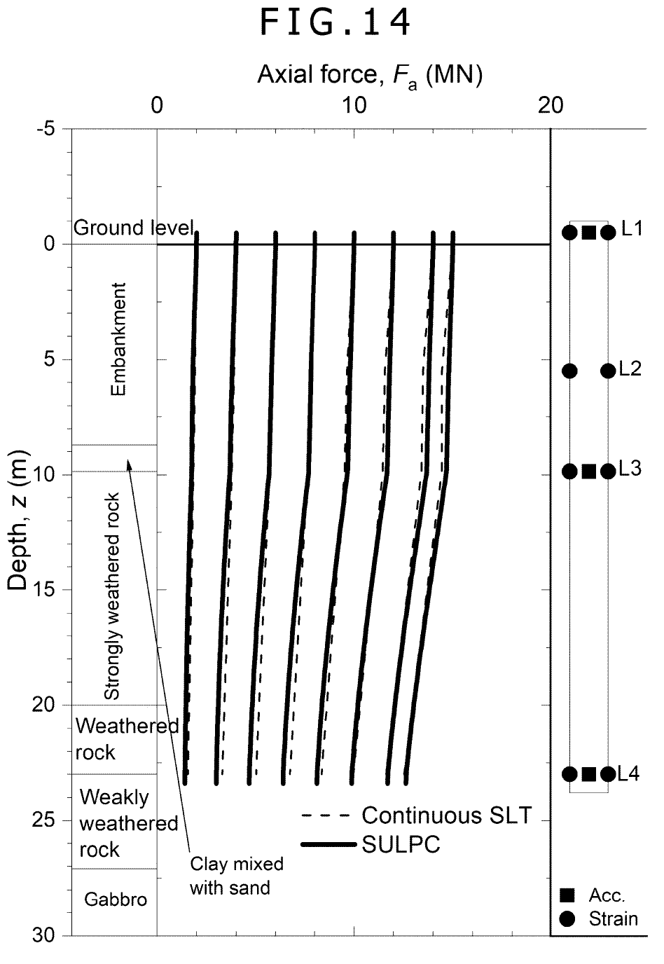

Further, FIG. 14 shows the axial force distributions obtained by use of the load transfer method. It is found that the axial force distributions obtained by use of the analysis method (SULPC method) in the present invention was well in agreement with the result obtained by use of the continuous load test method. This proves that the static soil resistance-pile displacement relation obtained by use of the analysis method (SULPC method) in the present invention and setting of the curve of the nonlinear spring model (see FIG. 12) are considered as being appropriate.

1.-2. (canceled)

3. A rapid load test method for a pile by use of a Segmental Unloading Point Connection method, in which a weight is dropped onto a pile head to determine a load-displacement relation of the pile from both of a load caused by drop of said weight and a displacement of said pile head, characterized in that a load-displacement relation is determined in accordance with the following steps on condition that a rapid load test is implemented on said pile head more than once in the manner of changing a drop height h of said weight, with said pile instrumented with strain gages and accelerometers at more than one depth level, the method comprises:
(1) step of, in said rapid load test implemented more than once, obtaining a rapid load F_{rapid}-pile displacement w relation on each pile segment, for each time of the rapid load test, using measurement data measured by said strain gages and accelerometers at more than one depth level of said pile,
(2) step of, for each time of the rapid load test, obtaining a soil resistance Rₛₒᵢₗ-pile displacement w relation on each pile segment, followed by the processing of calculating an unloading point that can be a point of maximum displacement of the pile, together with an unloading load R_{ULP} at that moment,
(3) step of obtaining a static soil resistance R_{w}-pile displacement w relation on each pile segment by use of an Unloading Point Connection method, using the calculated unloading point and unloading point load R_{ULP} for each time of the rapid load test in said step (2), followed by the processing of performing modeling of said static soil resistance-pile displacement relation into a nonlinear model, and
(4) step of performing an analysis on a load-displacement relation by use of a load transfer method with respect to the whole pile, using the nonlinear model of the soil resistance on each pile segment in said step (3).

4. The rapid load test method for the pile by use of the Segmental Unloading Point Connection method according to claim 3, wherein an analysis on axial force distributions in said pile is performed simultaneously in said step (4) of performing the analysis on the load-displacement relation.
